(19) United States
(12) Patent Application Publication
(10) Pub. No.: US 2013/0102690 A1
(43) Pub. Date: Apr. 25, 2013

(54) COLLAGEN-POLYSACCHARIDE MATERIALS MIMICKING BLOOD VESSELS, TISSUES AND BONES FOR MEDICAL, PHARMACEUTICAL AND ORTHOPEDIC APPLICATIONS, AND PROCESSES FOR PRODUCING THE SAME

(71) Applicant: NITTA CASINGS INC., Bridgewater, NJ (US)

(72) Inventors: Richard E. BATTERSBY, Stockton, NJ (US); Eugene Goldfarb, Marlboro, NJ (US); David E. Mathews, Somerville, NJ (US); Michael T. Shedlock, Easton, PA (US); Norberto A. Guzman, East Brunswick, NJ (US)

(73) Assignee: NITTA CASINGS INC., Bridgewater, NJ (US)

(21) Appl. No.: 13/657,378

(22) Filed: Oct. 22, 2012

Related U.S. Application Data
(60) Provisional application No. 61/550,104, filed on Oct. 21, 2011.

(51) Int. Cl. C08L 1/08; A61P 31/00; A61P 1/10; A61P 5/00; G09B 23/30; A61P 25/24; A61P 23/00; A61P 29/00; C08L 1/28; A61F 2/82; A61K 47/42; A61P 7/10

(52) U.S. Cl. 514/773; 106/143.1; 106/140.3; 106/139.3; 106/142.1; 623/1.15; 434/267; 434/272; 434/274

(57) ABSTRACT

Provided is a collagen-cellulose material containing, based on a total weight of the collagen-cellulose material: 1.0-9.0 wt. % of a collagen; 0.2-3.0 wt. % of cellulose or a derivative thereof; 0.5-6.5 wt. % of at least one acid selected from an inorganic acid, an organic acid, and mixtures thereof; and water. Also provided is a process for producing the collagen-cellulose material. Also provided is an artificial blood vessel containing the collagen-cellulose material which is in the form of a hollow tube, an artificial tissue containing the collagen-cellulose material which is in the form of a sheet, and an artificial bone containing the collagen-cellulose material which is in the form of a solid. Also provided is a medical device containing the collagen-cellulose material, as well as a process for testing phlebotomical, surgical or orthopedic instrumentation or practicing phlebotomical, surgical or orthopedic procedures using the medical device containing the collagen-cellulose material.

### BACKGROUND OF THE INVENTION

1. Field of the Invention

The present invention relates to a collagen-polysaccharide material which may be in the form of a hollow tube that mimics a blood vessel, a sheet that mimics a tissue, or a solid that mimics a bone for medical, pharmaceutical and orthopedic applications, as well as to a process for producing the collagen-polysaccharide material. The present invention also relates to an artificial blood vessel comprising the collagen-polysaccharide material which is in the form of a hollow tube, an artificial tissue comprising the collagen-polysaccharide material which is in the form of a sheet, and an artificial bone comprising the collagen-polysaccharide material which is in the form of a solid.

The present invention relates to a medical device comprising the collagen-polysaccharide material for testing phlebotomical, surgical or orthopedic instrumentation or practicing phlebotomical, surgical or orthopedic procedures. The present invention also relates to a process for testing phlebotomical, surgical or orthopedic instrumentation or practicing phlebotomical, surgical or orthopedic procedures using the medical device comprising the collagen-polysaccharide material.

The present invention also relates to art article comprising the collagen-polysaccharide material for pharmaceutical, medicinal, therapeutic, phlebotomical, surgical, or orthopedic applications.

2. Discussion of the Background

Collagen is a major fibrous protein constituent of cartilage, skin, bones, tendons, and other connective tissues of animals, as well as of skin, bones and scales of aquatic animals.

Collagen is present in all types of multicellular organisms and is probably the most abundant animal protein in nature. Collagen is located in the extracellular matrix of connective tissues and contributes to tissue integrity and mechanical properties.

Collagen molecules are composed of three polypeptide chains called alpha chains. The alpha chains contain about 1000 amino acid residues and, with the exception of short sequences at the ends of the chains, every third amino acid in each chain is glycine. The molecular formula of an alpha chain can thus be approximated as (X-Y-Gly)₃₃₃, where X and Y represent amino acids other than glycine. In collagen from mammals and birds about 100 of the X positions are proline, and about 100 of the Y positions are hydroxyproline. Collagen molecules have been classified into at least 29 types in the order in which they were purified and characterized, and grouped into classes according to their physicochemical properties.

Due to their excellent biocompatibility, biodegradability, and ease of extraction, purification and processing, collagen molecules have found use as a versatile biomaterial in numerous medical and pharmaceutical applications. Furthermore, collagen is used in the food and beverage industry, cosmetic industry, and nutraceutical industry in a variety of compositions.

Unembalmed anatomical materials simulating a “real world” tissue for testing phlebotomical, surgical or orthopedic instrumentation during research and development and/or for practicing phlebotomical, surgical or orthopedic procedures during the training of healthcare professionals (e.g., physicians, phlebotomists, surgeons, and medical students) suffer from a number of disadvantages including but not limited to a short shelf-life, rapid decomposition and the emission of foul odors, as well as the reliance on obtaining the unembalmed anatomical materials from animals and human cadavers.

Accordingly, there remains a critical need for a medical device in the form of an artificial blood vessel, an artificial tissue, and/or an artificial bone that is manufactured to replicate a naturally occurring blood vessel, tissue, and bone for testing phlebotomical, surgical or orthopedic instrumentation during research and development and/or for practicing phlebotomical, surgical or orthopedic procedures during the training of healthcare professionals (e.g., physicians, phlebotomists, surgeons, and medical students) while obviating the above-identified problems.

### SUMMARY OF THE INVENTION

The present invention provides a collagen-cellulose material which may be in the form of a hollow tube that mimics a blood vessel, a sheet that mimics a tissue, or a solid that mimics a bone for medical, pharmaceutical and orthopedic applications, as well as a process for producing the collagen-cellulose material. The present invention also provides an artificial blood vessel comprising the collagen-cellulose material which is in the form of a hollow tube, an artificial tissue comprising the collagen-cellulose material which is in the form of a sheet, and an artificial bone comprising the collagen-cellulose material which is in the form of a solid.

The present invention provides a medical device comprising the collagen-cellulose material for testing phlebotomical, surgical or orthopedic instrumentation or practicing phlebotomical, surgical or orthopedic procedures. The present invention also provides a process for testing phlebotomical, surgical or orthopedic instrumentation or practicing phlebotomical, surgical or orthopedic procedures using the medical device comprising the collagen-cellulose material.

The present invention also provides an article comprising the collagen-cellulose material for pharmaceutical, medicinal, therapeutic, phlebotomical, surgical, or orthopedic applications.

An exemplary aspect of the present invention is to provide a collagen-cellulose material comprising: a collagen; cellulose or a derivative thereof; at least one acid selected from an inorganic acid, an organic acid, and mixtures thereof; and water.

An exemplary aspect of the present invention is to provide a collagen-cellulose material comprising: 1.0-9.0 wt. % of a collagen, based on a total weight of the collagen-cellulose material; 0.2-3.0 wt. % of cellulose or a derivative thereof, based on the total weight of the collagen-cellulose material; 0.5-6.5 wt. % of at least one acid selected from an inorganic acid, an organic acid, and mixtures thereof, based on the total weight of the collagen-cellulose material; and water.

An exemplary aspect of the present invention is to provide a collagen-cellulose material comprising: 3.0-7.0 wt. % of a collagen, based on a total weight of the collagen-cellulose material; 0.8-2.0 wt. % of cellulose or a derivative thereof, based on the total weight of the collagen-cellulose material; 1.0-6.0 wt. % of at least one acid selected from an inorganic acid, an organic acid, and mixtures thereof, based on the total weight of the collagen-cellulose material; and water.

An exemplary aspect of the present invention is to provide a collagen-cellulose material comprising: 3.0-7.0 wt. % of a collagen, based on a total weight of the collagen-cellulose material; 0.8-2.0 wt. % of cellulose or a derivative thereof, based on the total weight of the collagen-cellulose material; 1.0-6.0 wt. % of at least one acid selected from an inorganic acid, an organic acid, and mixtures thereof, based on the total weight of the collagen-cellulose material; 0.1-25 wt. % of one or more compounds selected from pectin, sodium citrate, ammonium sulfate, citric acid, a crosslinking agent (e.g., glutaraldehyde and/or hydroxyacetaldehyde), an aluminum salt, a sugar, a sugar derivative obtained from a caramelization process, a sugar mixture comprising a sugar or a derivative thereof and a crosslinking agent (e.g., MAILLOSE®), a browning agent, glycerin, an antimicrobial agent (e.g., sodium benzoate and/or hypochlorite), mineral oil, poly(lactic-co-glycolic acid) (PLGA), hydroxyapatite, a surfactant, a plasticizer, a dye, and mixtures thereof, based on the total weight of the collagen-cellulose material; and water.

The collagen-cellulose material may be in the form of a hollow tube that mimics a blood vessel (e.g., an artery, a vein, or a capillary), a sheet (e.g., a slab, a pad, a membrane, or a film) that mimics a tissue, or a solid that mimics a bone. The hollow tube may have any desired dimension, including any desired length, aspect ratio, inner diameter, and/or outer diameter. The sheet may have any desired dimension, including any desired length, width, and/or thickness. The solid may have any desired dimension, including any desired shape, length, width, thickness, and/or outer diameter. The hollow tube, the sheet, and the solid may also have a desired tensile strength, modulus of elasticity, flexibility, porosity, compressibility, decompressibility, hardness, degree of crosslinking, wetness, sealability, and/or stability.

The foregoing discussion exemplifies certain aspects of the present invention. Additional exemplary aspects of the present invention are discussed in the following detailed description of the invention. The following description is to be regarded as illustrative in nature, and not as restrictive.

### DETAILED DESCRIPTION OF THE INVENTION

Unless specifically defined, all technical and scientific terms used herein have the same meaning as commonly understood by a skilled artisan in the relevant technological field.

The materials, processes and examples described herein are for illustrative purposes only and are therefore not intended to be limiting, unless otherwise specified.

Where a closed or open-ended numerical range is described herein, all values and subranges within or encompassed by the numerical range are to be considered as being specifically included in and belonging to the original disclosure of the present application as if these values and subranges had been explicitly written out in their entirety.

The meaning of each of the transitional phrases “comprising”, “consisting essentially of”, and “consisting of in the context of the present invention is as defined in MPEP §2111.03.

Recitations of “a” or “an” element, article or limitation within the claims and the specification is appropriately construed, and understood in the context of the present invention, to mean “one or more” unless otherwise specified.

The present invention provides a collagen-cellulose material comprising: a collagen; cellulose or a derivative thereof; at least one acid selected from an inorganic acid, an organic acid, and mixtures thereof; and water.

The present invention provides a collagen-cellulose material comprising: 1.0-9.0 wt. % of a collagen, based on a total weight of the collagen-cellulose material; 0.2-3.0 wt. % of cellulose or a derivative thereof, based on the total weight of the collagen-cellulose material; 0.5-6.5 wt. % of at least one acid selected from an inorganic acid, an organic acid, and mixtures thereof, based on the total weight of the collagen-cellulose material; and water.

The present invention provides a collagen-cellulose material comprising 1.0-9.0 wt. % of a collagen, including for example 1.5-8.5 wt. %, 2.0-8.0 wt. %, 2.5-7.5 wt. %, 3.0-7.0 wt. %, 3.5-6.5 wt. %, 4.0-6.0 wt. %, 4.5-5.5 wt. %, or 5.0 wt. %, based on a total weight of the collagen-cellulose material. The collagen is preferably present in an amount of 4.0-6.0 wt. %, 3.5-5.5 wt. %, or 3.0-5.0 wt. %, based on a total weight of the collagen-cellulose material. The collagen is particularly preferably present in an amount of 3.5-5.5 wt. %, based on a total weight of the collagen-cellulose material.

The present invention provides a collagen-cellulose material comprising 0.2-3.0 wt. % of cellulose or a derivative thereof, including for example 0.4-2.8 wt. %, 0.6-2.6 wt. %, or 0.8-2.4 wt. %, 1.0-2.2 wt. %, 1.2-2.0 wt. %, 1.4-1.8 wt. %, or 1.6 wt. %, based on a total weight of the collagen-cellulose material. The cellulose or the derivative thereof is preferably present in an amount of 0.6-2.2 wt. %, 0.8-2.0 wt. %, 1.0-1.8 wt. %, 1.2-1.6 wt. %, or 1.4 wt. %, based on a total weight of the collagen-cellulose material. The cellulose or the derivative thereof is particularly preferably present in an amount of 0.8-2.0 wt. %, based on a total weight of the collagen-cellulose material.

The cellulose may be obtained/derived from any plant source or material (e.g., wood). A particularly preferred source of cellulose is pectin-rich plant materials including for example peel (especially albedo, the white portion) of citrus fruits (especially lemon, lime, orange, and grapefruit), apple pomace, sugar beet pulp, sunflower heads, carrots, potatoes, tomatoes, and combinations thereof. For example, lemon peel contains approximately 2-4 wt. % of pectin when fresh and 20-40 wt. % of pectin when dried, whereas dried apple pomace contains approximately 10-20 wt. % of pectin. Cellulose obtained/derived from wood and/or orange peel is particularly preferred. Cellulose obtained/derived from orange peel is especially preferred because of a lower viscosity and a higher pectin content relative to that of wood.

The cellulose may exist in one or more various forms including for example powdered cellulose, microcrystalline cellulose, cellulose fibers, microfibrillated cellulose, and mixtures thereof. The microfibrillated cellulose is a nanocellulose material composed of nanosized cellulose fibrils having a high aspect ratio with a typical width of 5-20 nm and a typical length of up to 2,000 nm.

The derivative of cellulose may be an ether derivative of cellulose, non-limiting examples of which include methylcellulose, ethylcellulose, ethylmethylcellulose, hydroxyethylcellulose, hydroxyethylmethylcellulose, hydroxypropylcellulose, hydroxypropylmethylcellulose, ethylhydroxyethylcellulose, carboxymethylcellulose, carboxyethylcellulose, and mixtures thereof

The derivative of cellulose may be an ester derivative of cellulose, non-limiting examples of which include cellulose acetate, cellulose triacetate, cellulose propionate, cellulose acetate propionate, cellulose acetate butyrate, cellulose nitrate, cellulose sulfate, and mixtures thereof The cellulose component of the collagen-cellulose material of the present invention may be partially substituted with, or completely replaced by, one or more polysaccharides other than cellulose. Non-limiting examples of the one or more polysaccharides other than cellulose include alginate, α-glucan, amylopectin, amylose, arabinoxylan, β-glucan, carrageenan, chitin, chitosan, cyclodextrin, dextran, dextrin, fructan, gellan gum, glucan, glycogen, hemicellulose, inulin, maltodextrin, pectin, starch, and xanthan gum. Preferred collagen-polysaccharide materials of the present invention include a collagen-cellulose material, a collagen-alginate material, and mixtures thereof. A collagen-cellulose material represents a particularly preferred collagen-polysaccharide material of the present invention.

The present invention provides a collagen-cellulose material comprising 0.5-6.5 wt. % of at least one acid selected from an inorganic acid, an organic acid, and mixtures thereof, including for example 1.0-6.0 wt. %, 1.5-5.5 wt. %, 2.0-5.0 wt. %, 2.5-4.5 wt. %, 3.0-4.0 wt. %, or 3.5 wt. %, based on a total weight of the collagen-cellulose material. The at least one acid is preferably present in an amount of 1.0-6.0 wt. %, 1.5-5.5 wt. %, 2.0-5.0 wt. %, 2.5-4.5 wt. %, or 3.0-4.0 wt. %, based on a total weight of the collagen-cellulose material. The at least one acid is particularly preferably present in an amount of 2.0-5.0 wt. %, 2.5-4.5 wt. %, or 3.0-4.0 wt. %, based on a total weight of the collagen-cellulose material.

The acid may be one or more inorganic acids. Non-limiting examples of the inorganic acid include hydrochloric acid, nitric acid, phosphoric acid, sulfuric acid, boric acid, hydrofluoric acid, hydrobromic acid, perchloric acid, and mixtures thereof. Preferred inorganic acids include hydrochloric acid, phosphoric acid, sulfuric acid, and mixtures thereof. A particularly preferred inorganic acid is hydrochloric acid.

The acid may be one or more organic acids. Non-limiting examples of the organic acid include formic acid, acetic acid, propionic acid, butyric acid, valeric acid, caproic acid, and mixtures thereof Preferred organic acids include formic acid, acetic acid, propionic acid, and mixtures thereof. A particularly preferred organic acid is acetic acid.

The acid may be a mixture of one or more inorganic acids and one or more organic acids. A weight ratio of the one or more inorganic acids to the one or more organic acids includes for example 10/1 to 1/10, 9/1 to 1/9, 8/1 to 1/8, 7/1 to 1/7, 6/1 to 1/6, 5/1 to 1/5, 4/1 to 1/4, 3/1 to 1/3, 2/1 to 1/2, and 1/1. An inorganic acid to organic acid weight ratio of 9/2 to 2/9, 8/3 to 3/8, 7/4 to 4/7, 6/5 to 5/6, and 1/1 is preferred. An inorganic acid to organic acid weight ratio of 1/1 is particularly preferred. The acid is preferably a mixture of hydrochloric acid and acetic acid in a weight ratio of 3/1 to 1/3, 2/1 to 1/2, or 1/1.

The present invention provides a collagen-cellulose material comprising: 3.0-7.0 wt. % of a collagen, based on a total weight of the collagen-cellulose material; 0.8-2.0 wt. % of cellulose or a derivative thereof, based on the total weight of the collagen-cellulose material; 1.0-6.0 wt. % of at least one acid selected from an inorganic acid, an organic acid, and mixtures thereof, based on the total weight of the collagen-cellulose material; and water.

An exemplary aspect of the present invention is to provide a collagen-cellulose material comprising: 3.0-7.0 wt. % of a collagen, based on a total weight of the collagen-cellulose material; 0.8-2.0 wt. % of cellulose or a derivative thereof, based on the total weight of the collagen-cellulose material; 1.0-6.0 wt. % of at least one acid selected from an inorganic acid, an organic acid, and mixtures thereof, based on the total weight of the collagen-cellulose material; and a balance being water, wherein the total weight of the collagen, the cellulose or a derivative thereof, the at least one acid, and water is 100 wt. %.

The collagen-cellulose material may further comprise one or more compounds. Non-limiting examples of the one or more compounds include for example pectin, sodium citrate, ammonium sulfate, citric acid, a crosslinking agent (e.g., glutaraldehyde and/or hydroxyacetaldehyde), an aluminum salt, a sugar, a sugar derivative obtained from a caramelization process, a sugar mixture comprising a sugar or a derivative thereof and a crosslinking agent (e.g., MAILLOSE®), a browning agent, glycerin, an antimicrobial agent (e.g., sodium benzoate and/or hypochlorite), mineral oil, poly(lactic-co-glycolic acid) (PLGA), hydroxyapatite, a surfactant, a plasticizer, a dye, and mixtures thereof.

The one or more compounds are preferably selected from pectin, sodium citrate, ammonium sulfate, citric acid, a crosslinking agent (e.g., glutaraldehyde and/or hydroxyacetaldehyde), an aluminum salt, a sugar, a sugar derivative obtained from a caramelization process, a sugar mixture comprising a sugar or a derivative thereof and a crosslinking agent (e.g., MAILLOSE®), a browning agent, glycerin, sodium benzoate, and mixtures thereof.

The one or more compounds may be present in the collagen-cellulose material in an individual or combined amount of 0.1-25 wt. %, including for example 1.0-25.0 wt. %, 1.5-24.5 wt. %, 2.0-24.0 wt. %, 2.5-23.5 wt. %, 3.0-23.0 wt. %, 3.5-22.5 wt. %, 4.0-22.0 wt. %, 4.5-21.5 wt. %, 5.0-21.0 wt. %, 5.5-20.5 wt. %, 6.0-20.0 wt. %, 6.5-19.5 wt. %, 7.0-19.0 wt. %, 7.5-18.5 wt. %, 8.0-18.0 wt. %, 8.5-17.5 wt. %, 9.0-17.0 wt. %, 9.5-16.5 wt. %, 10.0-16.0 wt. %, 10.5-15.5 wt. %, 11.0-15.0 wt. %, 11.5-14.5 wt. %, 12.0-14.0 wt. %,12.5-13.5 wt. %, 13.0 wt. %, based on a total weight of the collagen-cellulose material.

Pectin may be present in an amount 0.1-5.0 wt. %, including for example 0.5-4.5 wt. %, 1.0-4.0 wt. %, 1.5-3.5 wt. %, 2.0-3.0 wt. %, or 2.5 wt. %, based on a total weight of the collagen-cellulose material.

The crosslinking agent (e.g., glutaraldehyde and/or hydroxyacetaldehyde) may be present in an amount of 50-500 ppm, including for example 100-450 ppm, 150-400 ppm, 200-350 ppm, or 250-300 ppm, based on a total weight of the collagen-cellulose material. Applicants have discovered that when pectin and/or an aluminum salt is combined with a crosslinking agent, crosslinking is enhanced and the hardness of the collagen hollow tubes and the collagen sheets more closely resembles that of blood vessels and tissue, respectively. Applicants have also discovered that when pectin and/or an aluminum salt is/are combined with a crosslinking agent and hydroxyapatite, crosslinking is enhanced and the hardness of the collagen solid more closely resembles that of bone.

The aluminum salt may be one or more aluminum salts selected from aluminum acetate, aluminum acetotartrate, aluminum acetylacetonate, aluminum bis(acetylsalicylate), aluminum borate. aluminum bromate, aluminum bromide, aluminum chlorate, aluminum chloride, aluminum citrate, aluminum fluoride, aluminum formate, aluminum iodide, aluminum lactate, aluminum maleate, aluminum nitrate, aluminum octoate, aluminum oleate, aluminum palmitate, aluminum phosphate, aluminum salicylate, aluminum stearate, aluminum sulfate, aluminum tartrate, aluminum tri(sec-butoxide), and mixtures thereof.

The sugar may be one or more sugars selected from a monosaccharide, a disaccharide, and mixtures thereof Non-limiting examples of the monosaccharide include glucose, fructose, galactose, ribose, arabinose, xylose, lyxose, allose, altrose, mannose, gulose, iodose, talose, and mixtures thereof. Non-limiting examples of the disaccharide include sucrose, lactulose, lactose, maltose, cellobiose, kojibiose, nigerose, isomaltose, trehalose, α,α-trehalose, β,β-trehalose, α,β-trehalose, sophorose, laminaribiose, gentiobiose, turanose, maltulose, palatinose, gentiobiose, mannobiose, melibiose, rutinose, rutinulose, xylobiose, and mixtures thereof

The sugar derivative obtained from a caramelization process may be present in a sugar mixture further comprising a crosslinking agent (e.g., hydroxyacetaldehyde, a.k.a., glycolaldehyde). MAIL LOSE® is a particularly preferred sugar mixture and/or browning agent. Applicants have discovered that the specific composition of the collagen-cellulose material can be adjusted (e.g., increasing or decreasing the concentration of one or more compounds within the collagen-cellulose material including, but not limited to, pectin, glutaraldehyde, aluminum, sugar, hydroxyapetite) in order to exhibit a desired tensile strength, modulus of elasticity, flexibility, porosity, compressibility. decompressibility (e.g., an ability to return to at least 80% of the original position), hardness, degree of crosslinking, wetness, sealability (e.g., an ability to maintain a hermetic seal following exposure to heat and/or pressure), and/or stability (e.g., a shelf life of at least six months with no change in physicochemical properties), as well as to mimic specific features of a desired anatomical structure.

The collagen-cellulose material may comprise less than 1.0 wt. % of calcium or a calcium compound (e.g., calcium oxide (a.k.a., lime), or calcium hydroxide (a.k.a., hydrated lime, caustic lime, slaked lime)), including for example less than 0.5 wt. %, less than 0.1 wt. %, less than 0.01 wt. %, and less than 0.001 wt. %, based on a total weight of the collagen-cellulose material. The collagen-cellulose material is particularly preferably decalcified and thus does not comprise calcium or a calcium compound.

The present invention provides a collagen-cellulose material comprising: 3.0-7.0 wt. % of a collagen, based on a total weight of the collagen-cellulose material; 0.8-2.0 wt. % of cellulose or a derivative thereof, based on the total weight of the collagen-cellulose material; 1.0-6.0 wt. % of at least one acid selected from an inorganic acid, an organic acid, and mixtures thereof, based on the total weight of the collagen-cellulose material; 0.1-25 wt. % of one or more compounds selected from pectin, sodium citrate, ammonium sulfate, citric acid, a crosslinking agent (e.g., glutaraldehyde and/or hydroxyacetaldehyde), an aluminum salt, a sugar, a sugar derivative obtained from a caramelization process, a sugar mixture comprising a sugar or a derivative thereof and a crosslinking agent (e.g., MAILLOSE®), a browning agent, glycerin, an antimicrobial agent (e.g., sodium benzoate and/or hypochlorite), mineral oil, poly(lactic-co-glycolic acid) (PLGA), hydroxyapatite, a surfactant, a plasticizer, a dye, and mixtures thereof, based on the total weight of the collagen-cellulose material; and water.

The present invention provides a collagen-cellulose material comprising: 3.0-7.0 wt. % of a collagen, based on a total weight of the collagen-cellulose material; 0.8-2.0 wt. % of cellulose or a derivative thereof, based on the total weight of the collagen-cellulose material; 1.0-6.0 wt. % of at least one acid selected from an inorganic acid, an organic acid, and mixtures thereof, based on the total weight of the collagen-cellulose material; 0.1-25 wt. % of one or more compounds selected from pectin, sodium citrate, ammonium sulfate, citric acid, a crosslinking agent (e.g., glutaraldehyde and/or hydroxyacetaldehyde), an aluminum salt, a sugar, a sugar derivative obtained from a caramelization process, a sugar mixture comprising a sugar or a derivative thereof and a crosslinking agent (e.g., MAILLOSE®), a browning agent, glycerin, an antimicrobial agent (e.g., sodium benzoate and/or hypochlorite), mineral oil, poly(lactic-co-glycolic acid) (PLGA), hydroxyapatite, a surfactant, a plasticizer, a dye, and mixtures thereof, based on the total weight of the collagen-cellulose material; and a balance being water, wherein the total weight of the collagen, the cellulose or a derivative thereof, the at least one acid, the one or more compounds, and water is 100%.

The collagen-cellulose material may be in the form of a hollow tube that mimics a blood vessel (e.g., an artery, a vein, or a capillary), a sheet (e.g., a slab, a pad, a membrane, or a film) that mimics a tissue (e.g., connective tissue, epidermal tissue, muscle tissue, or nerve tissue), or a solid that mimics a bone (e.g., femur bone, tooth). The hollow tube, the sheet, and the solid may also have a desired tensile strength, modulus of elasticity, flexibility, porosity, compressibility, decompressibility, hardness, degree of crosslinking, wetness, sealability, and/or stability.

The hollow tube may have any desired dimension, including any desired length, inner diameter, and/or outer diameter. Although the hollow tube generally has a circular cross-sectional shape, the hollow tube may have any desired cross-sectional shape including for example irregular, elliptical, triangular, square, pentagonal, hexagonal, etc. The hollow tube may be in the form of a bundle of a plurality of hollow tubes. The hollow tube may have any desired length including for example from 1.0 cm to 100 m. The hollow tube may have any desired internal and/or external diameter including for example from 0.10 mm to 50.0 mm. The hollow tube may represent an artificial blood vessel including an artificial artery, an artificial vein, or an artificial capillary.

The sheet may have any desired dimension, including any desired length, width and/or thickness. The sheet may have a rectangular cross-sectional shape of any desired dimension. The sheet may have any desired length, width and/or thickness including for example from 0.10 mm to 100 m, The sheet may represent an artificial tissue including an artificial connective tissue (e.g., an artificial tendon, an artificial ligament, or an artificial organ), epidermal tissue (e.g., artificial skin), muscle tissue, or nerve tissue.

The solid may have any desired dimension, including any desired shape, length, width, thickness, and/or outer diameter. The solid is generally in the shape of a bone (e.g., femur bone, tooth) and thus may represent an artificial bone.

The present invention also provides a process for producing the collagen-cellulose material, which may be in the form of a hollow tube, a sheet, or a solid. The hollow tubes, sheets, and solid of the collagen-cellulose material of the present invention may be prepared by a variation of a procedure used for the fabrication of collagen casings.

The collagen may be extracted/isolated from any source (e.g., any animal sources) including for example human, equine, bovine, porcine, alligator, and/or fish. The collagen is preferably extracted from bovine, porcine, and/or fish due to reduced antigenicity or an absence of antigenicity when the collagen-cellulose material is introduced in vivo.

In an exemplary aspect of the present invention, the collagen is extracted/isolated from the epidermis and/or the corium/dermis layer, which is the fibrous inner layer of the skin just beneath the epidermis and consists essentially of an enriched-collagen tissue, of an animal hide, preferably a bovine or porcine animal hide. The extracted/isolated collagen may be subjected to a limed split process which involves exposing the extracted/isolated collagen to calcium or a calcium compound (e.g., calcium oxide (a.k.a., lime), or calcium hydroxide (a.k.a., hydrated lime, caustic lime, slaked lime)) to breakdown the collagen into a fibrous structure to obtain a collagen split.

The collagen split may be subjected to decalcification with washing to remove undesirable calcium or calcium compounds, which have been incorporated into the collagen split during the limed split process, to obtain a purified collagen tissue, composed primarily of collagen fibers and collagen fibrils. Extensive washing may be carried out to remove a higher concentration of undesirable calcium or calcium compounds to thereby obtain collagen tissue having a higher degree of purity.

The purified collagen tissue may be subjected to acidification with a buffer composition, which comprises citric acid and/or a salt thereof (e.g., sodium citrate), ammonium sulfate, and water, to obtain a collagen material at a pH of 3.0-6.0, including for example 3.5-5.5, 4.0-5.0, or 4.5, preferably 4.0-5.0, 4.1-4.9, 4.2-4.8, 4.3-4.7, 4.4-4.6, or 4.5, and most preferably 4.5.

The collagen material may be cut and/or ground, optionally at a temperature of 10-25° C., including for example 11-24° C., 12-23° C., 13-22° C., 14-21° C., 15-20° C., 16-19° C., or 17-18° C., preferably 11-19° C., 14-18° C., 13-17° C., 12-16° C., or 13-15° C., and most preferably 13-17° C., 12-16° C., or 13-15° C., to obtain collagen which may be in the form of a powder, a fibril, and/or a fiber.

The collagen is mixed with: cellulose or a derivative thereof; at least one acid selected from an inorganic acid, an organic acid, and mixtures thereof; one or more optional compounds (e.g., a crosslinking agent (e.g., glutaraldehyde) and/or an aluminum salt); and water, for a time period of approximately 10-20 minutes, including for example 11-19 minutes, 12-18 minutes, 13-17 minutes, 14-16 minutes, or preferably 15 minutes, to form a fibrous slurry or a doughy mass having a pH of less than or equal to 3.0, including for example 1.0-3.0, 1.5-2.5, or 2.0.

Important steps in the formation of a fibrous slurry or a doughy mass having an appropriate consistency include the specific nature and concentrations of the components, the temperature, the pH, and/or the physical mixing/blending of the components.

The fibrous slurry or the doughy mass may be subjected to one or more treatments selected from cutting, grinding, homogenization, filtration (e.g., through a 0.008 mesh screen and/or a 0.006 mesh screen), dipping, soaking and/or extrusion (e.g., in the presence of an aluminum salt solution and/or a sugar mixture comprising a sugar or a derivative thereof and a crosslinking agent (e.g., MAILLOSE®)), washing (e.g., washing with an aqueous solution which may comprise an optional crosslinking agent, such as glutaraldehyde, for example), and/or drying.

The aluminum salt solution for use in the dipping, soaking and/or extrusion may comprise one or more of the aforementioned aluminum salts and an optional crosslinking agent (e.g., glutaraldehyde). A non-limiting example of the aluminum salt solution comprises 2.9-3.5 wt. % of an ammonium salt, 1.0-1.6 wt. % of ammonium sulfate, 0.4-1.0 wt. % of citric acid, 0.05-0.15 wt. % of ammonium hydroxide, and 225-275 ppm of glutaraldehyde. The fibrous slurry or the doughy mass may be soaked for a period of time (e.g., 30 minutes to 3.5 hours) in the aluminum salt solution prior to extrusion and/or be exposed to the aluminum salt solution during dipping and/or extrusion.

The sugar mixture comprising a sugar or a derivative thereof and a crosslinking agent (e.g., MAILLOSE®) may further comprise glycerin/glycerol and water. The fibrous slurry or the doughy mass may be soaked for a period of time (e.g., 30 minutes to 3.5 hours) in the sugar mixture prior to extrusion and/or be exposed to the sugar mixture during dipping and/or extrusion.

Following the treatment, the fibrous slurry or the doughy mass is subjected to aging in a storage tank at a temperature of less than 25° C. for a period of time (e.g., at least 6, 7, 8, 9, 10, 11, or 12 hours) sufficient to obtain a collagen-cellulose gel which is maintained at a temperature of less than 25° C. prior to being subjected to an extrusion process to obtain a collagen-cellulose material. The aluminum salt solution and/or the sugar mixture may be added to the storage tank at various concentrations in order to obtain a desired degree of crosslinking prior to being subjected to the extrusion process. In addition, or as an alternative, to adding the aluminum salt solution and/or the sugar mixture to the storage tank, the collagen-cellulose gel may be soaked for a period of time (e.g., 30 minutes to 3.5 hours) in the aluminum salt solution and/or the sugar mixture prior to extrusion and/or be exposed to the aluminum salt solution and/or the sugar mixture during extrusion.

The collagen-cellulose material obtained from the extrusion may be of any desired shape, length, width, thickness, inner diameter, and/or outer diameter, and may be in the form of a hollow tube, a sheet, or a solid depending on the specific type of extruder utilized during the extrusion process and the specific extrusion conditions (e.g., temperature, pressure, rate of extrusion, etc.). The collagen-cellulose material may be subjected to optional drying after the extrusion to obtain a desired moisture content and degree of wetness. The composition and manufacturing conditions of collagen-cellulose material is adjusted and designed/fabricated to exhibit a desired tensile strength, modulus of elasticity, flexibility, porosity, compressibility, decompressibility (e.g., an ability to return to at least 80% of the original position), hardness, degree of crosslinking, wetness, sealability (e.g., an ability to maintain a hermetic seal following exposure to heat and/or pressure), and/or stability (e.g., a shelf life of at least six months with no change in physicochemical properties), as well as to mimic specific features of a desired anatomical structure.

The following Table is a non-limiting comparison of various physicochemical properties of the collagen-cellulose material in the form of a hollow tube, a sheet, and a solid.

TABLE

| PROPERTY | TUBE | SHEET | SOLID |
|---|---|---|---|
| Tensile strength | Particularly | Particularly | Acceptable |
| | preferred | preferred | |
| Modulus of | Particularly | Acceptable | Acceptable |
| elasticity | preferred | | |
| Flexibility | Particularly | Acceptable | Acceptable |
| | preferred | | |
| Porosity | Particularly | Acceptable | Acceptable |
| | preferred | | |
| Compressibility | Particularly | Particularly | Acceptable |
| | preferred | preferred | |
| Decompressibility | Particularly | Particularly | Acceptable |
| | preferred | preferred | |
| Hardness | Acceptable | Particularly | Acceptable |
| | | preferred | |
| Degree of | Preferred | Particularly | Acceptable |
| Crosslinking | | preferred | |
| Wetness | Acceptable | Particularly | Acceptable |
| | | preferred | |
| Scalability | Particularly | Particularly | Acceptable |
| | preferred | preferred | |
| Stability | Particularly | Particularly | Particularly |
| | preferred | preferred | preferred |

The collagen-cellulose material obtained from the process of the present invention comprises: 1.0-9.0 wt. % of a collagen, based on a total weight of the collagen-cellulose material; 0.2-3.0 wt. % of cellulose or a derivative thereof, based on the total weight of the collagen-cellulose material; 0.5-6.5 wt. % of at least one acid selected from an inorganic acid, an organic acid, and mixtures thereof, based on the total weight of the collagen-cellulose material; and water.

The collagen-cellulose material obtained from the process of the present invention may be kept wet in a solution comprising: 5-15 wt. %, preferably 10 wt. % of glycerin; and 0.01-1.0 wt. %, preferably 0.1 wt. % of sodium benzoate.

The extracted/isolated collagen, the collagen-cellulose gel, and the collagen-cellulose material which may be in the form of a hollow tube, a sheet, or a solid, may be characterized by gel electrophoresis, capillary electrophoresis, and/or high-performance liquid chromatography, which may be carried out in the presence of sodium dodecyl sulfate. The degree of cross-linking of the collagen-cellulose gel and the collagen-cellulose material can also be determined.

The present invention also provides an artificial blood vessel including an artificial artery, an artificial vein, or an artificial capillary comprising the collagen-cellulose material which is in the form of a hollow tube.

The present invention also provides an artificial tissue including an artificial connective tissue (e.g., an artificial tendon, an artificial ligament, or an artificial organ), an artificial epidermal tissue (e.g., artificial skin), an artificial muscle tissue, or an artificial nerve tissue comprising the collagen-cellulose material which is in the form of a sheet.

The present invention also provides an artificial bone comprising the collagen-cellulose material which is in the form of a solid.

The present invention also provides a medical device comprising the collagen-cellulose material for testing phlebotomical, surgical or orthopedic instrumentation during research and development and/or for practicing phlebotomical, surgical or orthopedic procedures during the training of healthcare professionals (e.g., physicians, phlebotomists, surgeons, and medical students).

The medical device may further comprise a synthetic polymer, a natural polymer, an elastomer, a plastic, a thermoplastic, a synthetic rubber, a natural rubber, a silicone rubber, a hydrogel, a ceramic, a metal, an adhesive, a cement, wood, a dye, a pigment, an actual human blood vessel, bone or tissue, an actual animal blood vessel, bone or tissue, and combinations thereof.

The medical device comprising the collagen-cellulose material may be in the form of an artificial blood vessel (e.g., an artificial artery, an artificial vein, or an artificial capillary), an artificial bone (e.g., femur bone, tooth), or an artificial tissue including an artificial connective tissue (e.g., an artificial tendon, an artificial ligament, or an artificial organ), an artificial epidermal tissue (e.g., artificial skin), an artificial muscle tissue, or an artificial nerve tissue.

The medical device may further comprise a support member for supporting the artificial blood vessel, the artificial bone, and/or the artificial tissue. Non-limiting examples of the support member include a mannequin arm or an artificial arm for supporting the artificial blood vessel, a stand for supporting the artificial bone, and/or a tray for supporting the artificial tissue.

An exemplary aspect of the present invention is to provide a medical device comprising the collagen-cellulose material in the form of an artificial blood vessel, and a support member that may be in the form of a mannequin arm or an artificial arm for supporting the artificial blood vessel, which is useful for practicing phlebotomical procedures during the training of healthcare professionals (e.g., physicians, phlebotomists, surgeons, and medical students).

The medical device comprising the collagen-cellulose material of the present invention has a number of advantages including obviating the need to test or practice phlebotomical, surgical or orthopedic instrumentation and procedures on animals and human cadavers.

The present invention also provides a method for testing phlebotomical, surgical or orthopedic instrumentation and/or practicing a phlebotomical, surgical or orthopedic procedure comprising performing testing of the phlebotomical, surgical or orthopedic instrumentation or practicing of the phlebotomical, surgical or orthopedic procedure on a medical device comprising the collagen-cellulose material of the present invention.

The present invention also provides an article or device for in vitro or in vivo use comprising the collagen-cellulose material for pharmaceutical, medicinal, therapeutic, phlebotomical, surgical, or orthopedic applications.

The article for in vitro or in vivo use may further comprise a drug (e.g., antibiotic, analgesic, antihistamine, stimulant, tranquilizer, narcotic, antidepressant, hormone, anticoagulant, laxative, diuretic, etc.) and/or a pharmaceutically acceptable excipient. The article may be in any form including for example a powder, a pill, a tablet, a capsule, a liquid, a solution, a suspension, a slurry, a drink, a syrup, a thin film or membrane, a bandage, a transdermal patch, a suppository, a cream, a gel, a liniment, a balm, a lotion, an ointment, and a salve.

The device (e.g., an orthopedic device) for in vitro or in vivo use comprises the collagen-cellulose material in a form selected from a prosthetic blood vessel (e.g., a prosthetic artery, a prosthetic vein, or a prosthetic capillary), a prosthetic bone (e.g., a prosthetic femur bone, a prosthetic tooth), and/or a prosthetic tissue including prosthetic connective tissue (e.g., prosthetic tendon, prosthetic ligament, or prosthetic organ), prosthetic epidermal tissue (e.g., prosthetic skin), prosthetic muscle tissue, and/or prosthetic nerve tissue.

The collagen-cellulose material which is in the form of a hollow tube may be useful as a guide, a stent protector, a nerve protector, and for various biochemical mechanisms.

Numerous modifications and variations on the present invention are obviously possible in light of the above disclosure and thus the present invention may be practiced otherwise than as specifically described herein without departing from sprit and scope of the present invention. Accordingly, it is therefore to be understood that the foregoing disclosure is merely illustrative of exemplary aspects of the present invention and that numerous modifications and variations can be readily made by skilled artisans that fall within the scope of the accompanying claims.

1. A collagen-cellulose material comprising:
1.0-9.0 wt. % of a collagen, based on a total weight of the collagen-cellulose material;
0.2-3.0 wt. % of cellulose or a derivative thereof, based on the total weight of the collagen-cellulose material;
0.5-6.5 wt. % of at least one acid selected from the group consisting of an inorganic acid, an organic acid, and mixtures thereof, based on the total weight of the collagen-cellulose material; and
water.

2. The collagen-cellulose material according to claim 1, comprising: ‘3.0-7.0 wt. % of the collagen;
0.8-2.0 wt. % of the cellulose or the derivative thereof; and
1.0-6.0 wt. % of the at least one acid.

3. The collagen-cellulose material according to claim 1, wherein the cellulose is present and is obtained from wood, and/or at least one pectin-rich plant material selected from the group consisting of a peel of a citrus fruit, apple pomace, sugar beet pulp, a sunflower head, a carrot, a potato, a tomato, and combinations thereof.

4. The collagen-cellulose material according to claim 1, wherein the derivative of cellulose is present and is at least one ether derivative of cellulose selected from the group consisting of methylcellulose, ethylcellulose, ethylmethylcellulose, hydroxyethylcellulose, hydroxyethylmethylcellulose, hydroxypropylcellulose, hydroxypropylmethylcellulose, ethylhydroxyethylcellulose, carboxymethylcellulose, carboxyethylcellulose, and mixtures thereof

5. The collagen-cellulose material according to claim 1, wherein the derivative of cellulose is present and is at least one ester derivative of cellulose selected from the group consisting of cellulose acetate, cellulose triacetate, cellulose propionate, cellulose acetate propionate, cellulose acetate butyrate, cellulose nitrate, cellulose sulfate, and mixtures thereof

6. The collagen-cellulose material according to claim 1, wherein the acid is one or more inorganic acids selected from the group consisting of hydrochloric acid, nitric acid, phosphoric acid, sulfuric acid, boric acid, hydrofluoric acid, hydrobromic acid, perchloric acid, and mixtures thereof

7. The collagen-cellulose material according to claim 1, wherein the acid is one or more organic acids selected from the group consisting of formic acid, acetic acid, propionic acid, butyric acid, valeric acid, caproic acid, and mixtures thereof

8. The collagen-cellulose material according to claim 1, wherein the acid is a mixture of one or more inorganic acids and one or more organic acids, wherein a weight ratio of the one or more inorganic acids to the one or more organic acids is 10/1 to 1/10.

9. The collagen-cellulose material according to claim 1, wherein the acid is a mixture of hydrochloric acid and acetic acid in a weight ratio of 3/1 to 1/3.

10. The collagen-cellulose material according to claim 1, further comprising: ‘0.1-25 wt. % of one or more compounds selected from the group consisting of pectin, sodium citrate, ammonium sulfate, citric acid, a crosslinking agent, an aluminum salt, a sugar, a sugar derivative obtained from a caramelization process, a sugar mixture, a browning agent, glycerin, an antimicrobial agent, hydroxyapatite, and mixtures thereof, based on the total weight of the collagen-cellulose material.

11. A process for producing the collagen-cellulose material according to claim 1, wherein the process comprises:
mixing the collagen, the cellulose or a derivative thereof, the at least one acid, and water to form a fibrous slurry or a doughy mass;
cutting, grinding and/or homogenizing, and filtering the fibrous slurry or the doughy mass prior to aging in storage tank to obtain a collagen-cellulose gel; and
extruding the collagen-cellulose gel to obtain the collagen-cellulose material.

12. The process according to claim 11, wherein one or more compounds selected from the group consisting of pectin, sodium citrate, ammonium sulfate, citric acid, a crosslinking agent, an aluminum salt, a sugar, a sugar derivative obtained from a caramelization process, a sugar mixture, a browning agent, glycerin, an antimicrobial agent, hydroxyapatite, and mixtures thereof, are included in said mixing.

13. The process according to claim 11,
wherein said mixing is carried out for a time period of 10-20 minutes and the fibrous slurry or the doughy mass has a pH of less than or equal to 3.0;
wherein said filtering is through a 0.008 mesh screen and/or a 0.006 mesh screen and said aging is carried out for a time period of at least 6 hours in the storage tank which is maintained at a temperature of less than 25° C.; and
wherein said extruding of the collagen-cellulose gel is carried out in a manner so as to obtain the collagen-cellulose material in any desired shape, dimension, length, width, thickness, aspect ratio, inner diameter, and/or outer diameter.

14. The process according to claim 11, further comprising, in the following order and prior to said mixing:
extracting/isolating collagen from an animal source to obtain extracted/isolated collagen;
subjecting the extracted/isolated collagen to a limed split process by exposing the extracted/isolated collagen to calcium or a calcium compound to breakdown the extracted/isolated collagen into a fibrous structure so as to obtain a collagen split;
decalcifying the collagen split by washing to remove the calcium or the calcium compound to obtain a purified collagen tissue predominately comprising collagen fibers and collagen fibrils;
acidifying the purified collagen tissue with a buffer composition comprising citric acid, sodium citrate, ammonium sulfate, and water, to obtain a collagen material; and
cutting and/or grinding the collagen material to obtain collagen which may be in the form of a powder, a fibril, and/or a fiber.

15. The process according to claim 14,
wherein the animal source is at least one animal source selected from the group consisting of bovine, porcine, alligator, fish, and combinations thereof;
wherein the calcium compound, if present in the limed split process, includes calcium oxide and/or calcium hydroxide;
wherein the collagen material obtained from said acidifying has a pH of 3.0-6.0; and
wherein said cutting and/or grinding is carried out at a temperature of 10-25° C.

16. The process according to claim 11, wherein the process produces the collagen-cellulose material in the form of a hollow tube, a sheet, or a solid having a desired tensile strength, modulus of elasticity, flexibility, porosity, compressibility, decompressibility, hardness, degree of crosslinking, wetness, sealability, and/or stability.

17. An artificial blood vessel comprising the collagen-cellulose material according to claim 1 which is in the form of a hollow tube, an artificial tissue comprising the collagen-cellulose material according to claim 1 which is in the form of a sheet, or an artificial bone comprising the collagen-cellulose material according to claim 1 which is in the form of a solid.

18. A medical device comprising:
the collagen-cellulose material according to claim 1, which is in a form of an artificial blood vessel, an artificial tissue, and/or an artificial bone; and
at least one component selected from the group consisting of a synthetic polymer, a natural polymer, an elastomer, a plastic, a thermoplastic, a synthetic rubber, a natural rubber, a silicone rubber, a hydrogel, a ceramic, a metal, an adhesive, a cement, wood, a dye, a pigment, an actual human blood vessel, bone or tissue, an actual animal blood vessel, bone or tissue, and combinations thereof.

19. A method for testing phlebotomical, surgical or orthopedic instrumentation and/or practicing a phlebotomical, surgical or orthopedic procedure, wherein the method comprises performing testing of the phlebotomical, surgical or orthopedic instrumentation or practicing of the phlebotomical, surgical or orthopedic procedure on the medical device according to claim 18.

20. A medical device comprising:
the collagen-cellulose material according to claim 1, which is in the form of an artificial blood vessel; and
a support member in the form of a mannequin arm or an artificial arm for supporting the artificial blood vessel.

21. A method for practicing a phlebotomical procedure, wherein the method comprises performing practicing of the phlebotomical procedure on the medical device according to claim 20 by a healthcare professional.

22. The method according to claim 21, wherein the healthcare professional is one or more healthcare professionals selected from the group consisting of a physician, a phlebotomist, a surgeon, and a medical student.

23. An article comprising:
the collagen-cellulose material according to claim 1;
a drug; and
an optional pharmaceutically acceptable excipient,
wherein the article is in the form of a powder, a pill, a tablet, a capsule, a liquid, a solution, a suspension, a slurry, a drink, a syrup, a thin film or membrane, a bandage, a transdermal patch, a suppository, a cream, a gel, a liniment, a balm, a lotion, an ointment, or a salve.

24. An orthopedic device comprising the collagen-cellulose material according to claim 1 in the form of a prosthetic blood vessel, a prosthetic bone, and/or a prosthetic tissue.
